(19) United States
(12) Patent Application Publication
(10) Pub. No.: US 2023/0395259 A1
(43) Pub. Date: Dec. 7, 2023

(54) INFORMATION PROCESSING METHOD

(71) Applicant: NEC Corporation, Minato-ku, Tokyo (JP)

(72) Inventors: Yuki KOSAKA, Tokyo (JP); Kenji Araki, Tokyo (JP); Makoto Yasukawa, Tokyo (JP)

(73) Assignee: NEC Corporation, Minato-ku, Tokyo (JP)

(21) Appl. No.: 18/022,728

(22) PCT Filed: Aug. 28, 2020
(86) PCT No.: PCT/JP2020/032721

§ 371 Date: Feb. 22, 2023

(51) Int. Cl. G16H 50/30; G16H 50/50

(52) U.S. Cl. CPC G16H 50/30; G16H 50/50

(57) ABSTRACT

An information processing apparatus 100 includes an input unit 121 and a generation unit 122. The input unit 121 receives input of a first assessment value representing assessment of a subject at a predetermined point of time and input of a second assessment value representing assessment of the subject after a predetermined time elapsed from the predetermined point of time. The first and second assessment values are values for each of an item of the Stroke Impairment Assessment Set (SIAS) and an item of a second index, different from the SIAS, for assessing the condition of a human body. The generation unit 122 generates a model for calculating the second assessment value with respect to the first assessment value for each item of the SIAS and the second index, on the basis of information representing a relationship between the item of the SIAS and the item of the second index.

### TECHNICAL FIELD

The present invention relates to an information processing method, an information processing apparatus, and a program.

### BACKGROUND ART

Injuries, illnesses, aging, and the like may cause impaired reduce functions of activities of daily living and cognition functions. In such cases, rehabilitation is performed in a rehabilitation facility for recovery of the function of activities of daily living and cognitive functions. In a rehabilitation facility, it is necessary to grasp the conditions of motor/cognitive functions related to the activities of daily living of a patient who performs rehabilitation. As an example of an index for measuring the condition of such a patient, the Functional Independence Measure (FIM) is used. The FIM is an index for measuring motor/cognitive functions related to the activities of daily living. For example, as illustrated in Patent Literature 1 and FIG. 1, the FIM consists of a total of eighteen items, including thirteen types of motor items and five types of cognitive items, and each item is assessed according to the degree of need for assistance in four-level or seven-level scale.

A rehabilitation facility needs to predict recovery of a patient in order to develop a rehabilitation plan for the patient and give information about future assistance to the patient and the patient's family. For this reason, for example, it is conceivable to predict future assessment of each item of the FIM from the current situation of a new patient, with reference to the cases representing the past patient rehabilitation outcomes. The FIM is an example as an index for measuring the condition of a human body of a patient, and it is also possible to predict assessment of items set to other indices, different from the FIM, for assessing the condition of a human body.

Here, as another index for assessing the condition of a human body, there is an index called Stroke Impairment Assessment Set (SIAS). The SIAS is a comprehensive assessment set for quantifying functional impairments caused by stroke, and as illustrated in FIG. 2, it consists of twenty-two items classified into nine types of functional impairments, each of which is assessed on a three-point or five-point scale. As similar to the FIM described above, the SIAS is also required to predict future assessment from the current situation of a new patient by referring to examples of the past patient rehabilitation outcomes.

Patent Literature 1: JP 2017-027476 A

### SUMMARY

However, since the SIAS includes as many as twenty-two items, it takes time to measure, which places a working load on the facility. For this reason, it is difficult to collect many cases of the SIAS, so that it is difficult to predict future assessment based on the past cases. As a result, there is a problem that it is difficult to accurately predict the SIAS. In addition, in not only the SIAS but also other indices for assessing the condition of a human body in which it is difficult to collect many cases, there is a problem that it is difficult to predict such indices with high accuracy.

Accordingly, an object of the present invention is to propose an information processing method, an information processing apparatus, and a program capable of solving the above-described problem, that is, it is difficult to accurately predict assessment of items of an index for assessing the condition of a human body.

An information processing method, according to one aspect of the present invention, is configured to include
receiving input of a first assessment value representing assessment of a subject at a predetermined point of time and input of a second assessment value representing assessment of the subject after a predetermined time elapsed from the predetermined point of time, the first assessment value and the second assessment value being values for each of an item of Stroke Impairment Assessment Set (SIAS) and an item of a second index, different from the SIAS, for assessing a condition of a human body; and
generating a model for calculating the second assessment value with respect to the first assessment value for each of the item of the SIAS and the item of the second index, on the basis of information representing a relationship between the item of the SIAS and the item of the second index.

Further, an information processing method, according to one aspect of the present invention, is configured to include
on the basis of information representing a relationship between an item of Stroke Impairment Assessment Set (SIAS) and an item of a second index, different from the SIAS, for assessing a condition of a human body, inputting a new first assessment value of each of the item of the SIAS and the item of the second index, to a model generated so as to calculate a second assessment value representing assessment of a subject after a predetermined time elapsed from a predetermined point of time with respect to a first assessment value representing assessment of the subject at the predetermined point of time for each of the item of the SIAS and the item of the second index, and outputting a value calculated in the model in response to the input of the new first assessment value.

Further, an information processing apparatus, according to one aspect of the present invention, is configured to include
an input unit that receives input of a first assessment value representing assessment of a subject at a predetermined point of time and input of a second assessment value representing assessment of the subject after a predetermined time elapsed from the predetermined point of time, the first assessment value and the second assessment value being values for each of an item of Stroke Impairment Assessment Set (SIAS) and an item of a second index, different from the SIAS, for assessing a condition of a human body; and
a generation unit that generates a model for calculating the second assessment value with respect to the first assessment value for each of the item of the SIAS and the item of the second index, on the basis of information representing a relationship between the item of the SIAS and the item of the second index.

Further, an information processing apparatus, according to one aspect of the present invention, is configured to include
an input unit that, on the basis of information representing a relationship between an item of Stroke Impairment Assessment Set (SIAS) and an item of a second index, different from the SIAS, for assessing a condition of a human body, inputs a new first assessment value of each of the item of the SIAS and the item of the second index, to a model generated so as to calculate a second assessment value representing assessment of a subject after a predetermined time elapsed from a predetermined point of time with respect to a first assessment value representing assessment of the subject at the predetermined point of time for each of the item of the SIAS and the item of the second index; and
a prediction unit that outputs a value calculated in the model in response to the input of the new first assessment value.

Further, a program, according to one aspect of the present invention, is configured to cause an information processing apparatus to implement
an input unit that receives input of a first assessment value representing assessment of a subject at a predetermined point of time and input of a second assessment value representing assessment of the subject after a predetermined time elapsed from the predetermined point of time, the first assessment value and the second assessment value being values for each of an item of Stroke Impairment Assessment Set (SIAS) and an item of a second index, different from the SIAS, for assessing a condition of a human body; and
a generation unit that generates a model for calculating the second assessment value with respect to the first assessment value for each of the item of the SIAS and the item of the second index, on the basis of information representing a relationship between the item of the SIAS and the item of the second index.

Further, a program, according to one aspect of the present invention, is configured to cause an information processing apparatus to implement:

an input unit that, on the basis of information representing a relationship between an item of Stroke Impairment Assessment Set (SIAS) and an item of a second index, different from the SIAS, for assessing a condition of a human body, inputs a new first assessment value of each of the item of the SIAS and the item of the second index, to a model generated so as to calculate a second assessment value representing assessment of a subject after a predetermined time elapsed from a predetermined point of time with respect to a first assessment value representing assessment of the subject at the predetermined point of time for each of the item of the SIAS and the item of the second index; and
a prediction unit that outputs a value calculated in the model in response to the input of the new first assessment value.

With the configurations as described above, the present invention is capable of accurately predicting assessment of items of an index for assessing the condition of a human body.

### BRIEF DESCRIPTION OF DRAWINGS

FIG. 1 is a diagram for explaining the FIM.

FIG. 2 is a diagram for explaining the SIAS.

FIG. 3 is a block diagram illustrating a configuration of an information processing apparatus according to the present invention.

FIG. 4 illustrates examples of mathematical expressions to be used for generating a model by the information processing apparatus disclosed in FIG. 3, according to a first exemplary embodiment of the present invention.

FIG. 5 illustrates the relationship between the SIAS items and the FIM items.

FIG. 6 illustrates the relationship between the items of the FIM.

FIG. 7 illustrates an example of an adjacency matrix included in the expression disclosed in FIG. 4.

FIG. 8 is a flowchart illustrating an operation of the information processing apparatus disclosed in FIG. 3.

FIG. 9 illustrates another example of an adjacency matrix included in the expression disclosed in FIG. 4.

FIG. 10 is a block diagram illustrating a hardware configuration of an information processing apparatus according to a second exemplary embodiment of the present invention.

FIG. 11 is a block diagram illustrating a configuration of the information processing apparatus according to the second exemplary embodiment of the present invention.

FIG. 12 is a block diagram illustrating another configuration of the information processing apparatus according to the second exemplary embodiment of the present invention.

FIG. 13 is a flowchart illustrating an operation of the information processing apparatus according to the second exemplary embodiment of the present invention.

FIG. 14 is a flowchart illustrating another operation of the information processing apparatus according to the second exemplary embodiment of the present invention.

### EXEMPLARY EMBODIMENTS

### First Exemplary Embodiment

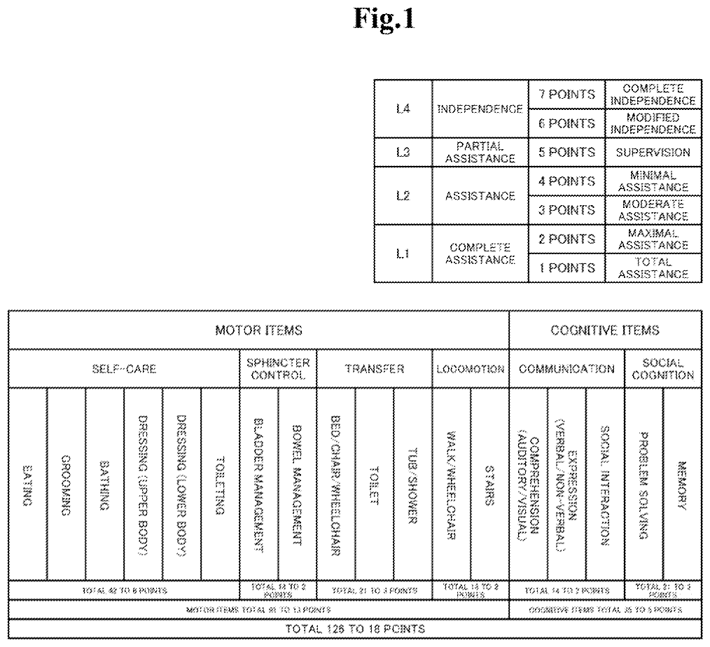

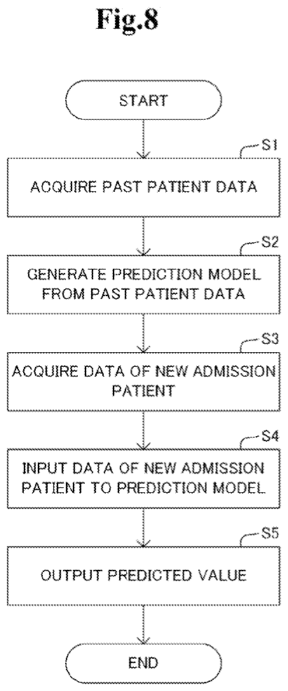

A first exemplary embodiment of the present invention will be described with reference to FIGS. 1 to 9. FIGS. 1 to 7 illustrate the configuration of an information processing apparatus, and FIG. 8 illustrates a processing operation of the information processing apparatus.

### Configuration

An information processing apparatus 10 is used to predict the future condition of a patient when the patient (subject) whose activities in daily living and cognitive functions have been deteriorated due to injury, illness, old age, or the like performs rehabilitation at a rehabilitation facility to recover activities in daily living and cognitive functions. Patients who are subject to rehabilitation include, but not limited to, patients with cerebrovascular diseases such as cerebral infarction and cerebral hemorrhage.

Specifically, it is assumed that the information processing apparatus 10 predicts an assessment value of at least one item set in the Stroke Impairment Assessment Set (SIAS) that is a comprehensive assessment set for quantifying functional impairment caused by stroke. In addition to the SIAS, the present embodiment uses at least one item of the Functional Independence Measure (FIM) that is an index for measuring motor/cognitive functions related to the patient's activities in daily living, to predict assessment values of the items of the SIAS and the FIM at the time of discharge from a facility in the future (after a predetermined time elapsed from admission), from the information of the patient including the assessment values of the items of the SIAS and the FIM at the time of admission to the facility (predetermined point of time). By predicting the assessment value of each SIAS item at the time of patient's discharge as described above, the facility can create an efficient rehabilitation plan for the patient. In addition, from the prediction result, it is possible to provide the patient and the patient's family with appropriate information regarding future assistance.

Note that the time of admission to a facility mentioned above is not necessarily limited to the date of admission, but may be any time when the SIAS and FIM items are assessed several days after the date of admission, or any other time that can be regarded as the time of admission in real terms. Further, the time of discharge from a facility mentioned above is not necessarily limited to the date of discharge, but may be the date when the patient is scheduled to be discharged from the admission date or when a predetermined period of time such as two weeks or one month has elapsed since the admission. Furthermore, the time of admission and the time of discharge described above are examples, and the information processing apparatus 10 may predict the assessment value of each item of the SIAS and the FIM at any later point of time based on the condition at any point of time during staying at the facility of the patient.

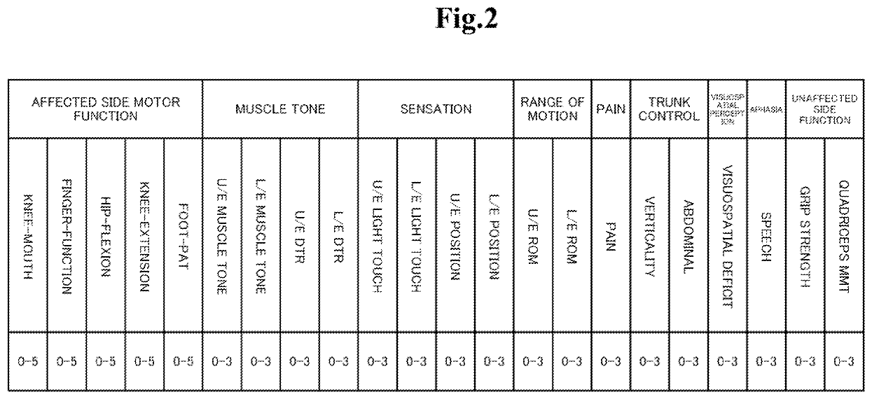

Here, the SIAS and the FIM mentioned above will be described in detail. First, the SIAS that is an index called the Stroke Impairment Assessment Scheme will be described with reference to FIG. 2. As illustrated in FIG. 2, the SIAS consists of twenty-two items classified into nine types of functional impairment, including “affected side motor function”, “muscle tone”, “sensation”, “range of motion”, “pain”, “trunk control”, “visuospatial perception”, “aphasia”, and “unaffected side function”. Specifically, in the SIAS, “affected side motor function” includes items such as “knee-mouth”, “finger-function”, “hip-flexion”, “knee-extension”, and “foot-pat”, “muscle tone” include items such as “U/E muscle tone”, “L/E muscle tone”, “U/E DTR”, and “L/E DTR”, “sensation” includes items such as “U/E light touch”, “L/E light touch”, “U/E position”, and “L/E position”, “range of motion” includes items such as “upper ROM” and “lower ROM”, “pain” includes an item such as “pain”, “trunk control” includes items such as “verticality” and “abdominal”, “visuospatial perception” includes an item such as “visuospatial deficit”, “aphasia” includes an item such as “speech”, and “unaffected side function” includes items such as “grip strength” and “quadriceps MMT”. Each of these twenty-two items will be assessed with an assessment value of 3-point or 5-point scale, as illustrated in FIG. 2.

Next, the FIM, that is an index for measuring the motor/cognitive functions related to the patients' activities of daily living will be described with reference to FIG. 1. As illustrated in FIG. 1, the FIM consists of a total of eighteen items, that is, thirteen motor items to assess the patient's “motor function” and five cognitive items to assess the patient's “cognitive function”. Specifically, the FIM includes, as the abovementioned motor items, items for assessing the patient's function of activities of a “self-care” category such as “eating”, “glooming”, “bathing”, “dressing (upper body)”, “dressing (lower body)” and “toileting”, items for assessing the patient's function of activities of a “sphincter control” category such as “bladder management” and “bowel management”, items for assessing the patient's function of activities of a “transfer” category such as “bed/chair/wheelchair”, “toilet” and “tub/shower”, and items for assessing the patient's function of activities of a “locomotion” category such as “walk/wheelchair” and “stairs”. Moreover, the FIM includes, as the cognitive items, items for assessing the patient's function of a “communication” category such as “comprehension (auditory/visual)” and “expression (verbal/non-vernal), and items for assessing the patient's function of a “social cognition” category such as “social interaction”, “problem solving” and “memory”.

With the FIM, a degree of assistance necessary for a patient is assessed on a four-level or seven-level scale for each of the aforementioned items. For example, as shown in the upper right part of FIG. 1, each item may be assessed by degrees of four-level scale such as “L1: complete dependence on helper”, “L2: helper”, “L3: modified dependence on helper”, and “L4: no helper”. Moreover, for example, each item may be assessed by degrees of seven-level scale such as “total assistance”, “maximal assistance”, “moderate assistance”, “minimal assistance, “supervision”, “modified independence”, and “complete independence”. In the case of such assessment on a seven-level scale, a patient may be assessed by aggregating levels given to the respective assessment degrees for each item, each category, and each function.

The assessment of the items of the SIAS and the FIM described above is usually performed by a specialist who assists the patient as an evaluator. For example, they are assessed by “occupational therapists”, “physical therapists”, “nurses”, “speech therapists”, and the like.

The assessment value of each of the SIAS and FIM items is input into a data management device 20 by an expert who is the above-mentioned evaluator, and is stored as patient data. For example, the data management device 20 stores patient data for each patient as an electronic medical record. The electronic medical record stores therein information such as “gender”, “age group”, “consciousness level (JCS: Japan Coma Scale), “disease name”, “paralysis condition”, “assessment value of each item of the SIAS and the FIM at admission (first assessment value)”, and “assessment value of each item of the SIAS and the FIM at discharge (second assessment value)”, for example, as patient data. However, patient data is not necessarily limited to including the information described above, but may include only some of the information described above, or may include different information. Note that the patient data of a patient still in the hospital does not include “assessment value of each item of the SIAS and the FIM at discharge”.

In the present invention, by using the patient data stored in the data management device 20 as described above, the information processing apparatus 10 predicts the assessment value of each item of the SIAS and the FIM at discharge, of a patient at admission or a patient who is recently admitted to the hospital. To this end, the information processing apparatus 10 has a configuration as described below, to realize the function of performing a process of generating a model for predicting an assessment value of each item of the SIAS and the FIM at discharge of a patient (model generation process) and a process of predicting the assessment value of each item of the SIAS and the FIM at discharge of the patient by using the generated model (prediction process).

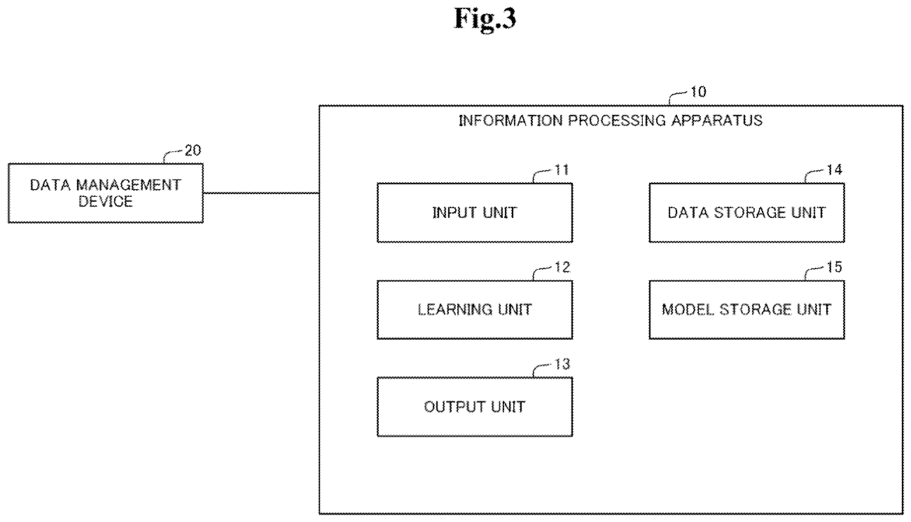

The information processing apparatus 10 is configured of one or more information processing apparatuses equipped with an arithmetic device and a memory device. As illustrated in FIG. 3, the information processing apparatus 10 includes an input unit 11, a learning unit 12, and an output unit 13, constructed by execution of a program by the arithmetic device. The information processing apparatus 10 also includes a data storage unit 14 and a model storage unit 15 that are formed in the storage device. Each constituent element will be described in detail below.

The input unit 11 requests the data management device 20 for patient data, receives input of such patient data, and stores it in the data storage unit 14. In the model generation process, the input unit 11 requests and acquires patient data of patients who have already been discharged from hospital, as learning data. For example, the input unit 11 requests patient data in which a flag indicating that the patient has been discharged is set, or patient data in which assessment values of the FIM items at the time of discharge have been input, and acquires it as learning data. The input unit 11 may acquire such patient data as learning data without requesting the data management device 20 for the patient data. For example, whenever the patient data of a patient who has already been discharged is updated in the data management device 20, the input unit 11 may acquire the patient data as learning data. In the prediction process, the input unit 11 requests patient data of a patient who has not been discharged from hospital and is a subject of the prediction process, as data for prediction. For example, the input unit 11 requests patient data in which a flag indicating that the patient has been discharged is not set, or patient data in which assessment values of the FIM items at the time of discharge have not been input, and acquires it as data for prediction. Patient data as data for prediction of a patient who is a subject of the prediction process is acquired after the model is generated as described below, but the timing of acquiring patient data is not limited thereto.

The learning unit 12 (generation unit) performs machine learning by using the patient data acquired as the learning data, generates a model for predicting an assessment value of each item of the FIM at the time of discharge of the patient, and stores the model in the model storage unit 15. At that time, the learning unit 12 generates, by machine learning, a model function represented by a function (f_i(X_n)) whose input value (X_n: n=1, . . . , N (N: number of patients)) is “basic information” such as “gender”, “age group”, “consciousness level”, “disease name”, and “paralysis condition” in the patient data and “information at admission” such as “assessment value of each item of the SIAS and the FIM at admission (first assessment value)”, and whose output value (y_i=1, . . . , 40 (items)) is “assessment value of each item of the SIAS and the FIM at discharge (second assessment values)”. That is, for each item of the SIAS and the FIM, the learning unit 12 generates a model function to calculate the output value (y_i) with respect to the input value (X_n). As described above, there are twenty-two SIAS items and eighteen FIM items, which means that a model function is generated for each of a total of forty items.

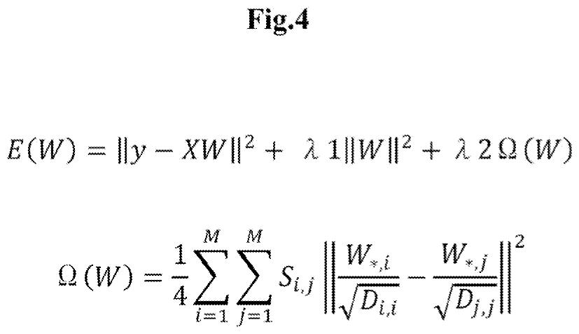

In the present embodiment, the learning unit 12 generates the model function f_i by using ridge regression. Specifically, the learning unit 12 generates the model function (f_i) by calculating a parameter (W) (coefficient) of each term constituting the model function (f_i) so that the assessment function (loss function) shown in the upper row of FIG. 4 is minimized.

At that time, in the present embodiment, as illustrated in the upper row of FIG. 4, an assessment function that includes two regularization terms with parameter a (W) is used. Specifically, the first regularization term is “λ1∥w∥²” and the second regularization term is “λ2Ω(W)”. In this case, λ1 and λ2 are parameters that adjust the degree of influence of the respective regularization terms on the loss function. It is assumed that these parameters are given in advance. As the magnitude of λ1 and λ2 is larger, the effect on the loss function is stronger.

Further, in the present embodiment, in particular, “Ω(W)” constituting the regularization term of the final term contains an adjacency matrix represented by “Sij”, as illustrated in the lower row of FIG. 4. The adjacency matrix “Si,j” is information that represents the relationship between a SIAS item and a FIM item. For example, “1” is set between mutually related items and “0” is set between mutually unrelated items.

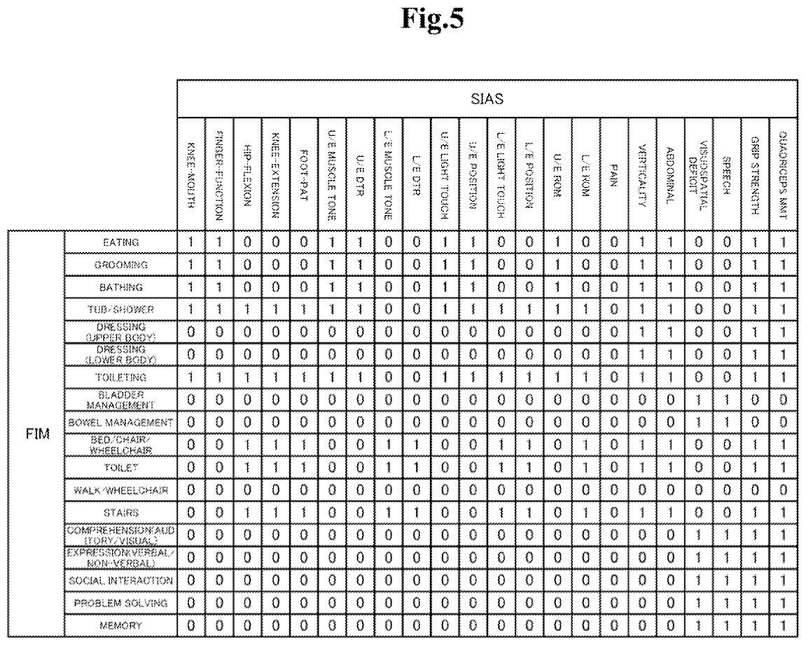

Here, the adjacency matrix “Si,j” will be described with reference to FIGS. 5 to 7. FIG. 5 illustrates the relationship between each SIAS item and each FIM item. For example, since the “grip strength” item in the SIAS and the “eating” item in the FIM have similarity in the assessment content, that is, there is a correlation, “1” is set between these items. When there is a correlation between the SIAS and the FIM in other items as well, “1” is set between such items. However, the relationship between each SIAS item and each FIM item illustrated in FIG. 5 is an example, and the relationship may be set according to other criteria.

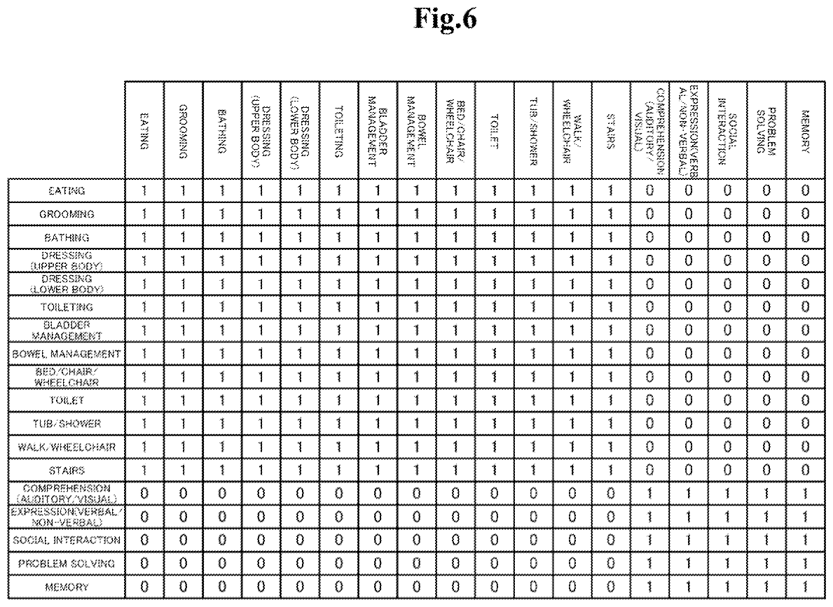

FIG. 6 illustrates the relationship between respective FIM items, and “1” set between items that have similarity in the assessment content of the respective FIM items, that is, there is a correlation. Specifically, in the example of FIG. 6, it is assumed that items in the FIM are associated with each other if they belong to the same “function” (“motor” or “cognitive”), and “1” is set between the items that belong to the “motor” function and between the items that belong to the “cognitive” function. However, the relationship between the respective FIM items illustrated in FIG. 6 is an example, and the relationship may be set according to other criteria.

Although not illustrated, the relationship between respective SIAS items is also set, and “1” is set between the items having similarity in the assessment contents of the respective SIAS items, that is, there is a correlation. For example, it is assumed that when the functions to which items in the SIAS belong are the same, the items are associated with each other, and “1” is set. Note that the relationship between the SIAS items may be set according to any criteria.

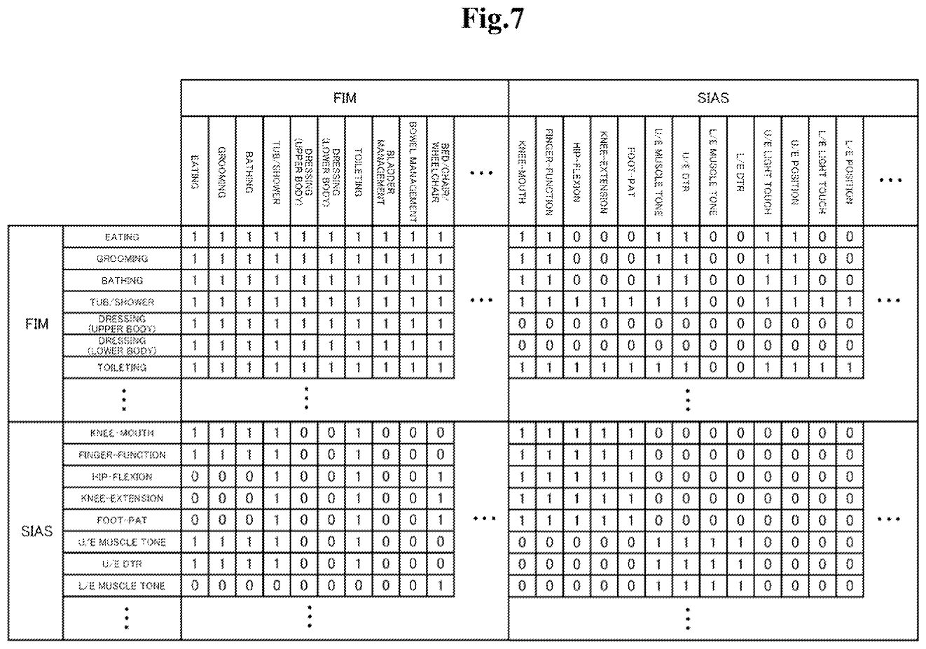

FIG. 7 illustrates an example of an adjacency matrix “Si,j” that combines the relationship between each SIAS item and each FIM item, the relationship between the SIAS items, and the relationship between the FIM items, in a single matrix. Since there are twenty-two SIAS items and eighteen FIM items, the total number of items is 40, so that the adjacency matrix “Si,j” is a 40 by 40 matrix.

In the present embodiment, by providing a regularization term including an adjacency matrix according to the relationship between the SIAS items and the FIM items as described above, a function (f_i) can be generated such that the parameters in the function (f_i) corresponding to the SIAS item and the FIM item that are associated with each other are similar to each other. In other words, in the expression shown in the lower row of FIG. 4, the difference between the parameters of the function corresponding to the SIAS item and the FIM item that are associated with each other are squared, and the parameters are optimized to be similar to each other to make the value of the assessment function smaller. Similarly, by providing a regularization term that includes an adjacency matrix corresponding to the relationship between the SIAS item and between the FIM item, the function (f_i) can be generated such that the parameters in the function (f_i) for the SIAS items that are associated with each other and the parameters in the function (f_i) for the FIM items that are associated with each other are similar to each other.

The regularization using the adjacency matrix as described above is described in the below literature and is an existing technology, so that the detailed explanation thereof is omitted.

Nozomi Nori, Hisashi Kashima, Kazuto Yamashita, Hiroshi Ikai, and Yuichi Imanaka, “Simultaneous Modeling of Multiple Diseases for Mortality Prediction in Acute Hospital Care” in Proceedings of the 21th ACM SIGKDD International Conference on Knowledge Discovery and Data Mining, pp. 855-864, 2015

The output unit 13 (prediction unit) inputs patient data of a patient who has not been discharged, acquired as prediction data by the input unit 11, to the model function (f_i) generated as described above. In other words, the output unit 13 inputs, to the model function, “basic information” such as “gender”, “age group”, “consciousness level”, “disease name”, and “paralysis condition” in the just admitted patient data and “information at admission” such as “assessment value of each item of the SIAS and the FIM at admission (first assessment value)” as an input value (X_n′), and calculates the output value (y_i′) from the model function (f_i(X_n′)). Thereby, it is possible to predict assessment value of each item of the SIAS and the FIM at discharge, for a newly admitted patient.

### Operation

Next, operation of the information processing apparatus 10 described above will be explained with reference to the flowchart of FIG. 8. First, the information processing apparatus 10 performs a model generation process to generate a model for predicting an assessment value of each item of the SIAS and the FIM at the time of patient discharge. Therefore, the information processing apparatus 10 requests the data management device 20 for the past patient data, and acquires the patient data as learning data (step S1).

Then, the information processing apparatus 10 generates, by machine learning, a model function represented by a function whose input values are “basic information” such as “gender”, “age group”, “consciousness level”, “disease name”, and “paralysis condition” in the patient data and “information at admission” such as “assessment value of each item of the SIAS and the FIM at admission” and whose output value is “assessment value of each item of the SIAS and the FIM at discharge” (step S2). At that time, the information processing apparatus 10 generates a model function by using ridge regression. In particular, as described above, the parameter of each term constituting the model function is optimized by using an assessment function with an additional regularization term that includes an adjacency matrix that is information representing the relationship between the SIAS item and the FIM items. Thereby, it is possible to generate a model function in which parameters in the model function corresponding to the mutually related SIAS item and FIM item are similar to each other.

Then, the information processing apparatus 10 performs a prediction process to predict an assessment value of each item of the SIAS and the FIM at the time of patient discharge, by using the generated model. Therefore, the information processing apparatus 10 requests the data management device 20 for patient data of a newly admitted patient or a patient who has been admitted but not yet discharged, and acquires such patient data as data for prediction (step S3). Note that the patient data acquired as data for prediction does not include an assessment value of each item of the SIAS and the FIM at the time of discharge because the patient has not been discharged.

Then, the information processing apparatus 10 inputs, to the model function, “basic information” such as “gender”, “age group”, “consciousness level”, “disease name”, and “paralysis condition” in the patient data and “information at admission” such as an “assessment value of each item of the SIAS and the FIM at admission” as input values (step S4). Then, the information processing apparatus 10 outputs an “assessment value of each item of the SIAS and the FIM at discharge” calculated by the model function, as a predicted value (step S5). Thereby, it is possible to predict an assessment value of each item of the SIAS and the FIM at discharge of an admitted patient. The output prediction result can then be used, for example, to create an efficient rehabilitation plan for the patient at the facility, or to provide advice to the patient and the patient's family regarding the future assistance.

As described above, according to the present invention, a model is generated to calculate an assessment value of each of the SIAS and the FIM while considering the relationship between the items of the SIAS and the FIM, based on the information of the previous patients who performed rehabilitation. As a result, it is possible to predict assessment of each item of the SIAS and the FIM at the time of discharge accurately and quickly, by using the assessment of other indices and the assessment of other items of the same index. In particular, in the above example, when predicting the assessment of each item of the SIAS at the time of discharge, it is possible to predict the assessment of each item of the SIAS with high accuracy by using the assessment of each item of the FIM that is easily measured and a large amount of data thereof can be easily collected.

Although the above example shows the case where an assessment value of each item of the SIAS and the FIM at discharge is predicted from the patient data at the time of admission of the patient, an assessment value of each item of the SIAS and the FIM thereafter may be predicted by using the patient data at any point of time during staying at the facility.

Further, while the assessment value of each item set in the SIAS and the FIM is used in the above description, the values of items set in other indices such as those used for assessing the condition of a human body may also be used. For example, an index used to assess the balance function of the elderly and stroke patients such as the Berg Balance Scale (BBS) may be used. The BBS has a total of fourteen items ranging from simple balance functions such as “postural retention” and “standing up movement” to advanced balance functions such as “functional reach test”, “tandem walking test”, and “one-leg standing test”, and each item is assessed with “0 to 4 points”.

Then, as similar to the above description, a model may be generated to calculate the assessment value of each item set in the SIAS and the BBS, and a predicted value of each item may be calculated. In that case, information indicating the relationship between the SIAS items and the BBS items is used. In other words, as similar to the information indicating the relationship between the SIAS items and the FIM items illustrated in FIG. 5, information indicating the relationship between the SIAS items and the BBS items is prepared in advance. Similarly, information indicating the relationship between the respective SIAS items and information indicating the relationship between the respective BBS items are also prepared. By using such information, it is possible to generate an adjacency matrix “Si,j” between thirty-six items, that is, twenty-two items from the SIAS and fourteen items from the BBS, as similar to the adjacency matrix “Si,j” illustrated in FIG. 7.

Then, by using the expression illustrated in FIG. 4 including the adjacency matrix “Si,j” described above, it is possible to generate, by machine learning, a model function whose input values are “basic information” in the patient data and “information at admission” such as “assessment value of each item of the SIAS and the BBS at admission” and whose output value is “assessment value of each item of the SIAS and the BBS at discharge”, as described above. Further, by inputting “basic information” of a new patient and “information at admission” such as “assessment value of each item of the SIAS and the BBS at admission” into the generated model function as input values, it is possible to output “assessment value of each item of the SIAS and the BBS at discharge”, calculated by the model function, as a predicted value.

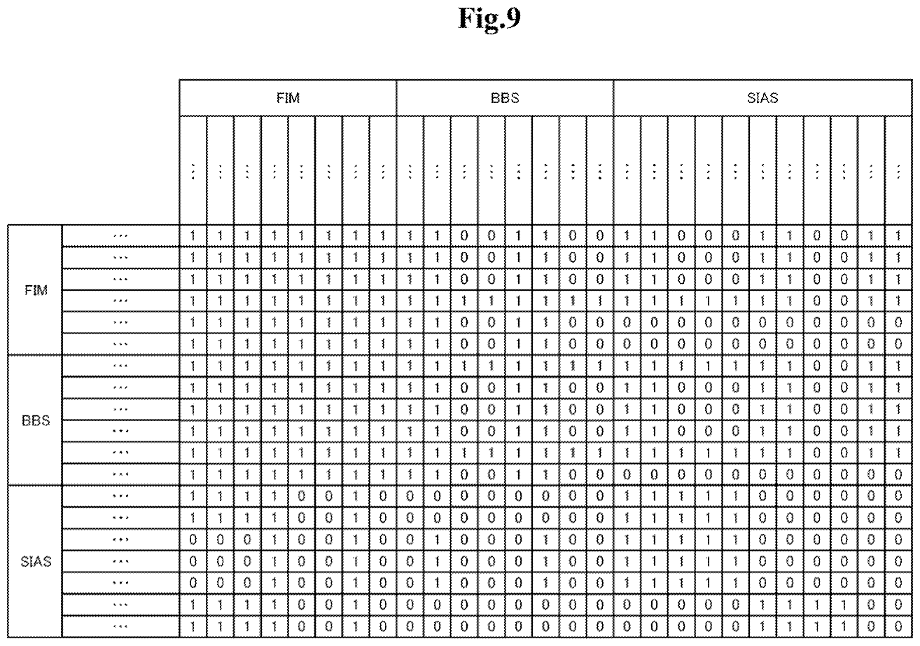

Moreover, it is possible to use assessment values of the three indices, namely the SIAS, the FIM, and the BBS described above, to generate a model to calculate an assessment value of each item set in the three indices, and calculate the predicted value of each item. That is, assessment values of respective items of the other two indices, that is, the FIM and the BBS, may be used to predict an assessment value of an item of the SIAS. In that case, information indicating the relationship between the SIAS items and the FIM items is used. In other words, in addition to the information indicating the relationship between the SIAS items and the FIM items illustrated in FIG. 5, information indicating the relationship between the SIAS items and the BBS items and information indicating the relationship between the FIM items and the BBS items are prepared in advance. Similarly, information indicating the relationship between the respective SIAS items, information indicating the relationship between the respective FIM items, and information indicating the relationship between the respective BBS items are prepared. By using such information, it is possible to generate an adjacency matrix “Si,j” between a total of fifty-four items, that is, twenty-two items of the SIAS, eighteen items of the FIM, and fourteen items of the BBS, as illustrated in FIG. 9.

Then, by using the expression illustrated in FIG. 4 including the adjacency matrix “Si,j” described above, it is possible to generate, by machine learning, a model function whose input values are “basic information” in the patient data and “information at admission” such as “assessment value of each item of the SIAS, the FIM, and the BBS at admission” and whose output value is “assessment value of each item of the SIAS, the FIM, and the BBS at discharge”, as described above. Further, by inputting “basic information” of a new patient and “information at admission” such as “assessment value of each item of the SIAS, the FIM, and the BBS at admission” into the generated model function as input values, it is possible to output “assessment value of each item of the SIAS, the FIM, and the BBS at discharge”, calculated by the model function, as predicted values.

Note that the present invention is not limited to be applicable to the above-mentioned indices such as the SIAS, the FIM, and the BBS, but may be applied to other indices for assessing the condition of a human body. In addition, while the cases where two or three indices are used have been described above, a larger number of indices may be used.

### Second Exemplary Embodiment

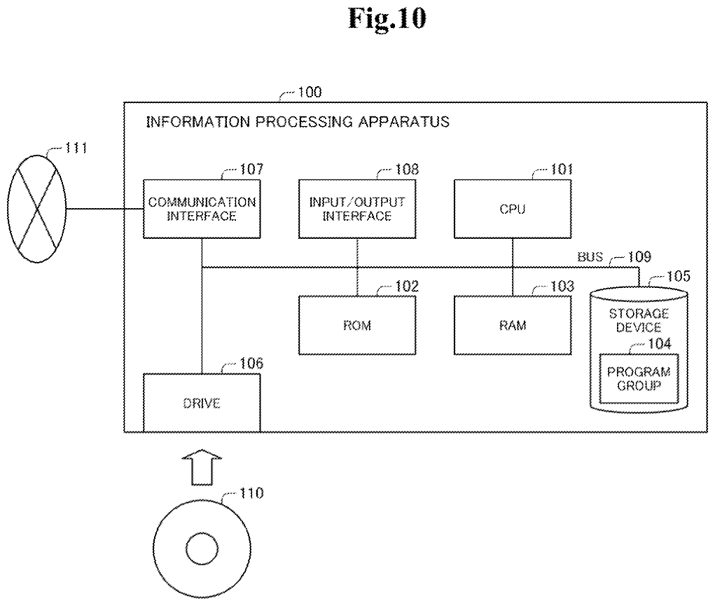

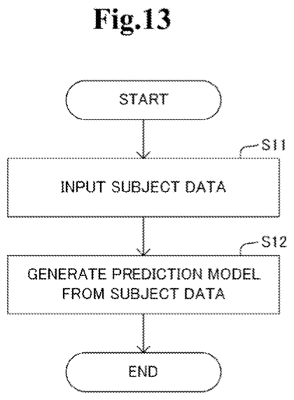

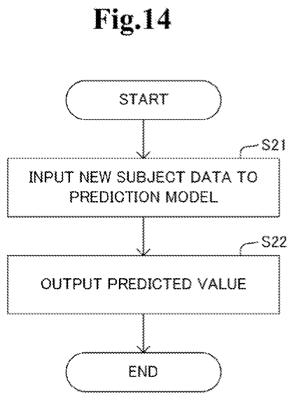

Next, a second exemplary embodiment of the present invention will be described with reference to FIGS. 10 to 14. FIGS. 10 to 12 are block diagrams illustrating the configuration of an information processing apparatus according to the second exemplary embodiment, and FIGS. 13 and 14 are flowcharts illustrating the operation of the information processing apparatus. The present embodiment illustrates the outlines of the configurations of the information processing apparatus and the information processing method described in the first exemplary embodiment.

First, a hardware configuration of an information processing apparatus 100 according to the present embodiment will be described with reference to FIG. 10. The information processing apparatus 100 is configured of a general information processing apparatus, and includes the following hardware configuration as an example:
Central Processing Unit (CPU) 101 (arithmetic device)
Read Only Memory (ROM) 102 (storage device)
Random Access Memory (RAM) 103 (storage device)
Program group 104 to be loaded to the RAM 103
Storage device 105 storing therein the program group 104
Drive 106 that performs reading and writing on a storage medium 110 outside the information processing apparatus
Communication interface 107 connecting to a communication network 111 outside the information processing apparatus
Input/output interface 108 for performing input/output of data
Bus 109 connecting the respective constituent elements

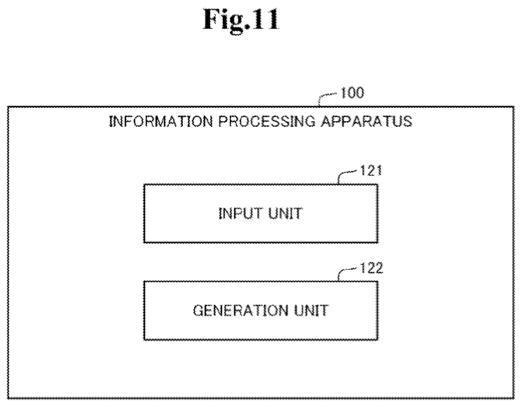

The information processing apparatus 100 can construct and can be equipped with an input unit 121 and a generation unit 122 illustrated in FIG. 11 through acquisition and execution of the program group 104 by the CPU 101. Note that the program group 104 is, for example, stored in the storage device 105 or the ROM 102 in advance, and is loaded to the RAM 103 by the CPU 101 as needed. Alternatively, the program group 104 may be provided to the CPU 101 via the communication network 111, or may be stored on a storage medium 110 in advance and read out by the drive 106 and provided to the CPU 101. However, the input unit 121 and the generation unit 122 may be constructed with electronic circuits.

Note that FIG. 10 shows an exemplary hardware configuration of the information processing apparatus 100, and the hardware configuration of the information processing apparatus is not limited to the case described above. For example, the information processing apparatus may be configured of part of the configuration described above, such as without the drive 106.

The information processing apparatus 100 executes the information processing method illustrated in the flowchart of FIG. 13 by the functions of the input unit 121 and the generation unit 122 that are constructed by the program as described above.

As illustrated in FIG. 13, the information processing apparatus 100

receives input of a first assessment value representing assessment of a subject at a predetermined point of time and input of a second assessment value representing assessment of the subject after a predetermined time elapsed from the predetermined point of time, the first assessment value and the second assessment value being values for each of an item of the Stroke Impairment Assessment Set (SIAS) and an item of a second index, different from the SIAS, for assessing a condition of a human body (step S11), and

generates a model for calculating the second assessment value with respect to the first assessment value for each of the item of the SIAS and the item of the second index, on the basis of information representing the relationship between the item of the SIAS and the item of the second index (step S12).

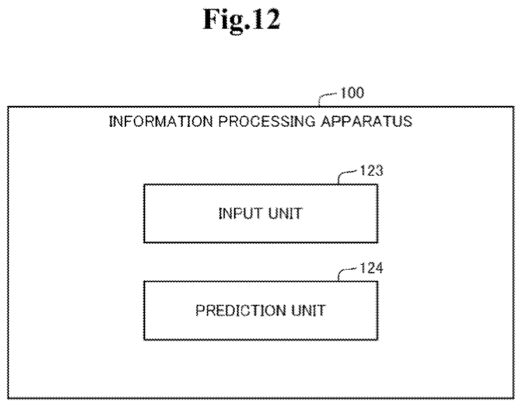

The information processing apparatus 100 can also construct and be equipped with an input unit 123 and a prediction unit 124 illustrated in FIG. 12 through acquisition and execution of the program group 104 by the CPU 101. However, the input unit 123 and the prediction unit 124 may be constructed with electronic circuits.

The information processing apparatus 100 executes the information processing method illustrated in the flowchart of FIG. 14 by the functions of the input unit 123 and the prediction unit 124 that are constructed by the program as described above.

As illustrated in FIG. 14, the information processing apparatus 100

inputs, on the basis of information representing the relationship between an item of the Stroke Impairment Assessment Set (SIAS) and an item of a second index, different from the SIAS, that assesses the condition of a human body, to a model generated by calculating a second assessment value representing the assessment of the subject after a predetermined time elapsed from a predetermined point of time with respect to a first assessment value representing the assessment of the subject at the predetermined point of time for each of the item of the SIAS and the item of the second index, a new first assessment value of each of the item of the SIAS and the item of the second index (step S21), and outputs a value calculated by the model corresponding to the input of the new first assessment value (step S22).

Note that the information processing apparatus 100 described above is configured of, for example, a server computer installed in a facility such as a hospital where the subject patient performs rehabilitation, or a server computer on the so-called cloud operated and managed by such a facility. The values calculated and output by the information processing apparatus 100 as described above are displayed on information processing terminals (personal computers, tablet terminals, smartphones, and the like) used by therapists, nurses, and other medical professionals who assist in the rehabilitation of the patient at the facility, and are referenced by the medical professionals.

Since the present embodiment is configured as described above, the present embodiment generates a model for calculating an assessment value of each item of the SIAS, while considering the relationship between an item of the SIAS and an item of another index. By using the relationship between an item of the SIAS and an item of another index, it is possible to predict an assessment value of each item accurately and quickly, even for an assessment index such as the SIAS in which data collection is difficult. The index to which the present invention is applicable is not limited to the SIAS, but is applicable to any other index for assessing the condition of a human body.

### Supplementary Note

The whole or part of the exemplary embodiments disclosed above can be described as the following supplementary notes. Hereinafter, outlines of an information processing method, an information processing apparatus, and a program of the present invention will be described. However, the present invention is not limited to the following configurations.

### Supplementary Note 1

An information processing method comprising:

receiving input of a first assessment value representing assessment of a subject at a predetermined point of time and input of a second assessment value representing assessment of the subject after a predetermined time elapsed from the predetermined point of time, the first assessment value and the second assessment value being values for each of an item of Stroke Impairment Assessment Set (SIAS) and an item of a second index, different from the SIAS, for assessing a condition of a human body; and

generating a model for calculating the second assessment value with respect to the first assessment value for each of the item of the SIAS and the item of the second index, on the basis of information representing a relationship between the item of the SIAS and the item of the second index.

### Supplementary Note 2

The information processing method according to supplementary note 1, further comprising

generating the model on the basis of information indicating whether or not the item of the SIAS and the item of the second index are associated with each other.

### Supplementary Note 3

The information processing method according to supplementary note 1 or 2, further comprising

generating the model on the basis of information in which the item of the SIAS and the item of the second index are associated with each other according to contents of assessment of the item of the SIAS and the item of the second index.

### Supplementary Note 4

The information processing method according to supplementary note 2 or 3, further comprising

generating the model such that parameters included in the model corresponding to the item of the SIAS and the item of the second index that are associated with each other become similar.

### Supplementary Note 5

The information processing method according to any of supplementary notes 2 to 4, further comprising

generating the model by using a loss function with an additional regularization term that includes an adjacency matrix representing the relationship between the item of the SIAS and the item of the second index.

### Supplementary Note 6

The information processing method according to any of supplementary notes 1 to 5, further comprising

receiving input of a value representing an assessment degree of the subject at the predetermined point of time for each of the item of the SIAS and the item of the second index as the first assessment value, and input of a value representing an assessment degree of the subject after the predetermined time elapsed from the predetermined point of time as the second assessment value.

### Supplementary Note 7

The information processing method according to any of supplementary notes 1 to 6, further comprising

inputting, to the model, a new first assessment value for each of the item of the SIAS and the item of the second index, and outputting a value calculated by the model in response to the input of the new first assessment value.

### Supplementary Note 8

The information processing method according to any of supplementary notes 1 to 7, further comprising

receiving input of the first assessment value and the second assessment value for each of the item of the SIAS and items of a plurality of the second indices that are different from each other; and

generating a model for calculating the second assessment value with respect to the first assessment value in each of the item of the SIAS and the items of the plurality of the second indices, on the basis of information representing a relationship between the item of the SIAS and the items of the plurality of the second indices.

### Supplementary Note 9

An information processing method comprising:

on the basis of information representing a relationship between an item of Stroke Impairment Assessment Set (SIAS) and an item of a second index, different from the SIAS, for assessing a condition of a human body, inputting a new first assessment value of each of the item of the SIAS and the item of the second index, to a model generated so as to calculate a second assessment value representing assessment of a subject after a predetermined time elapsed from a predetermined point of time with respect to a first assessment value representing assessment of the subject at the predetermined point of time for each of the item of the SIAS and the item of the second index, and outputting a value calculated in the model in response to the input of the new first assessment value.

### Supplementary Note 10

The information processing method according to any of supplementary notes 1 to 9, wherein

the second index is Functional Independence Measure (FIM).

### Supplementary Note 11

The information processing method according to any of supplementary notes 1 to 9, wherein

the second index is Berg Balance Scale (BBS).

### Supplementary Note 12

An information processing apparatus comprising:

an input unit that receives input of a first assessment value representing assessment of a subject at a predetermined point of time and input of a second assessment value representing assessment of the subject after a predetermined time elapsed from the predetermined point of time, the first assessment value and the second assessment value being values for each of an item of Stroke Impairment Assessment Set (SIAS) and an item of a second index, different from the SIAS, for assessing a condition of a human body; and

a generation unit that generates a model for calculating the second assessment value with respect to the first assessment value for each of the item of the SIAS and the item of the second index, on the basis of information representing a relationship between the item of the SIAS and the item of the second index.

### Supplementary Note 13

The information processing apparatus according to supplementary note 12, further comprising

a prediction unit that outputs a value calculated by the model in response to input, to the model, of a new first assessment value of each of the item of the SIAS and the item of the second index.

### Supplementary Note 14

An information processing apparatus comprising:

an input unit that, on the basis of information representing a relationship between an item of Stroke Impairment Assessment Set (SIAS) and an item of a second index, different from the SIAS, for assessing a condition of a human body, inputs a new first assessment value of each of the item of the SIAS and the item of the second index, to a model generated so as to calculate a second assessment value representing assessment of a subject after a predetermined time elapsed from a predetermined point of time with respect to a first assessment value representing assessment of the subject at the predetermined point of time for each of the item of the SIAS and the item of the second index; and

a prediction unit that outputs a value calculated in the model in response to the input of the new first assessment value.

### Supplementary Note 15

A computer-readable medium storing thereon a program for causing an information processing apparatus to implement:

an input unit that receives input of a first assessment value representing assessment of a subject at a predetermined point of time and input of a second assessment value representing assessment of the subject after a predetermined time elapsed from the predetermined point of time, the first assessment value and the second assessment value being values for each of an item of Stroke Impairment Assessment Set (SIAS) and an item of a second index, different from the SIAS, for assessing a condition of a human body; and

a generation unit that generates a model for calculating the second assessment value with respect to the first assessment value for each of the item of the SIAS and the item of the second index, on the basis of information representing a relationship between the item of the SIAS and the item of the second index.

### Supplementary Note 16

The computer-readable medium storing thereon the program according to supplementary note 15, for causing the information processing apparatus to further implement

a prediction unit that outputs a value calculated by the model in response to input, to the model, of a new first assessment value of each of the item of the SIAS and the item of the second index.

### Supplementary Note 17

A computer-readable medium storing thereon a program for causing an information processing apparatus to implement:

an input unit that, on the basis of information representing a relationship between an item of Stroke Impairment Assessment Set (SIAS) and an item of a second index, different from the SIAS, for assessing a condition of a human body, inputs a new first assessment value of each of the item of the SIAS and the item of the second index, to a model generated so as to calculate a second assessment value representing assessment of a subject after a predetermined time elapsed from a predetermined point of time with respect to a first assessment value representing assessment of the subject at the predetermined point of time for each of the item of the SIAS and the item of the second index; and

a prediction unit that outputs a value calculated in the model in response to the input of the new first assessment value.

### Supplementary Note 18

An information processing method comprising

receiving input of a first assessment value representing assessment of a subject at a predetermined point of time and input of a second assessment value representing assessment of the subject after a predetermined time elapsed from the predetermined point of time, the first assessment value and the second assessment value being values for each of an item of a first index for assessing a condition of a human body and an item of a second index, different from the first index, for assessing a condition of a human body; and

generating a model for calculating the second assessment value with respect to the first assessment value for each of the item of the first index and the item of the second index, on the basis of information representing a relationship between the item of the first index and the item of the second index.

### Supplementary Note 19

An information processing method comprising:

on the basis of information representing a relationship between an item of a first index for assessing a condition of a human body and an item of a second index, different from the first index, for assessing a condition of a human body, inputting a new first assessment value of each of the item of the first index and the item of the second index, to a model generated so as to calculate a second assessment value representing assessment of a subject after a predetermined time elapsed from a predetermined point of time with respect to a first assessment value representing assessment of the subject at the predetermined point of time for each of the item of the first index and the item of the second index, and outputting a value calculated in the model in response to the input of the new first assessment value.

The program described above can be stored using various types of non-transitory computer readable media and supplied to a computer. Non-transitory computer readable media include various types of tangible storage media. Examples of non-transitory computer readable media include magnetic recording media (e.g., flexible disks, magnetic tape, hard disk drives), magneto-optical recording media (e.g., magneto-optical disks), CD-ROM (Read Only Memory), CD-R, CD-R/W, and semiconductor memories (e.g., mask ROM, PROM (Programmable ROM), EPROM (Erasable PROM), flash ROM, and RAM (Random Access Memory)). The program may also be supplied to a computer by various types of transitory computer readable media. Examples of transitory computer readable media include electrical signals, optical signals, and electromagnetic waves. A transitory computer readable medium can supply the program to a computer via wired or wireless communication channels such as wires and fiber optics.

While the present invention has been described with reference to the exemplary embodiments and the like described above, the present invention is not limited to the above-described embodiments. The form and details of the present invention can be changed within the scope of the present invention in various manners that can be understood by those skilled in the art.

### REFERENCE SIGNS LIST

10 information processing apparatus

11 input unit

12 learning unit

13 output unit

14 data storage unit

15 model storage unit

20 data management device

100 information processing apparatus

101 CPU

102 ROM

103 RAM

104 program group

105 storage device

106 drive

107 communication interface

108 input/output interface

109 bus

110 storage media

111 communication network

121 input unit

122 generation unit

123 input unit

124 prediction unit

what is claimed is:

1. An information processing method comprising:
receiving input of a first assessment value representing assessment of a subject at a predetermined point of time and input of a second assessment value representing assessment of the subject after a predetermined time elapsed from the predetermined point of time, the first assessment value and the second assessment value being values for each of an item of Stroke Impairment Assessment Set (SIAS) and an item of a second index, different from the SIAS, for assessing a condition of a human body; and
generating a model for calculating the second assessment value with respect to the first assessment value for each of the item of the SIAS and the item of the second index, on the basis of information representing a relationship between the item of the SIAS and the item of the second index.

2. The information processing method according to claim 1, further comprising
generating the model on the basis of information indicating whether or not the item of the SIAS and the item of the second index are associated with each other.

3. The information processing method according to claim 1, further comprising
generating the model on the basis of information in which the item of the SIAS and the item of the second index are associated with each other according to contents of assessment of the item of the SIAS and the item of the second index.

4. The information processing method according to claim 2, further comprising
generating the model such that parameters included in the model corresponding to the item of the SIAS and the item of the second index that are associated with each other become similar.

5. The information processing method according to claim 2, further comprising
generating the model by using a loss function with an additional regularization term that includes an adjacency matrix representing the relationship between the item of the SIAS and the item of the second index.

6. The information processing method according to claim 1, further comprising
receiving input of a value representing an assessment degree of the subject at the predetermined point of time for each of the item of the SIAS and the item of the second index as the first assessment value, and input of a value representing an assessment degree of the subject after the predetermined time elapsed from the predetermined point of time as the second assessment value.

7. The information processing method according to claim 1, further comprising
inputting, to the model, a new first assessment value for each of the item of the SIAS and the item of the second index, and outputting a value calculated by the model in response to the input of the new first assessment value.

8. The information processing method according to claim 1, further comprising
receiving input of the first assessment value and the second assessment value for each of the item of the SIAS and items of a plurality of the second indices that are different from each other; and
generating a model for calculating the second assessment value with respect to the first assessment value for each of the item of the SIAS and the items of the plurality of the second indices, on the basis of information representing a relationship between the item of the SIAS and the items of the plurality of the second indices.

9. (canceled)

10. The information processing method according to claim 1, wherein
the second index is Functional Independence Measure (FIM).

11. The information processing method according to claim 1, wherein
the second index is Berg Balance Scale (BBS).

12. An information processing apparatus comprising:
at least one memory configured to store instructions; and
at least one processor configured to execute instructions to:
receive input of a first assessment value representing assessment of a subject at a predetermined point of time and input of a second assessment value representing assessment of the subject after a predetermined time elapsed from the predetermined point of time, the first assessment value and the second assessment value being values for each of an item of Stroke Impairment Assessment Set (SIAS) and an item of a second index, different from the SIAS, for assessing a condition of a human body; and
generate a model for calculating the second assessment value with respect to the first assessment value for each of the item of the SIAS and the item of the second index, on the basis of information representing a relationship between the item of the SIAS and the item of the second index.

13. The information processing apparatus according to claim 12, wherein the at least one processor is configured to execute the instructions to
output a value calculated by the model in response to input, to the model, of a new first assessment value of each of the item of the SIAS and the item of the second index.

14. (canceled)

15. A non-transitory computer-readable medium storing thereon a program comprising instructions for causing an information processing apparatus to execute instructions to:
receive input of a first assessment value representing assessment of a subject at a predetermined point of time and input of a second assessment value representing assessment of the subject after a predetermined time elapsed from the predetermined point of time, the first assessment value and the second assessment value being values for each of an item of Stroke Impairment Assessment Set (SIAS) and an item of a second index, different from the SIAS, for assessing a condition of a human body; and
generate a model for calculating the second assessment value with respect to the first assessment value for each of the item of the SIAS and the item of the second index, on the basis of information representing a relationship between the item of the SIAS and the item of the second index.

16. The non-transitory computer-readable medium storing thereon the program comprising the instructions according to claim 15, for causing the information processing apparatus to further execute processing to
output a value calculated by the model in response to input, to the model, of a new first assessment value of each of the item of the SIAS and the item of the second index.

17. (canceled)
